(19) United States
(12) Patent Application Publication
(10) Pub. No.: US 2022/0355541 A1
(43) Pub. Date: Nov. 10, 2022

(54) ADDITIVE MANUFACTURING OF HYDROGEL TUBES FOR BIOMEDICAL APPLICATIONS

(71) Applicant: Lung Biotechnology PBC, Silver Spring, MD (US)

(72) Inventors: Mora Carolynne Melican, Weston, MA (US); Lara Murcin, Stratham, NH (US); Richmon Lin, Silver Spring, MD (US); Derek Morris, Bedford, NH (US); Barbara Nsiah, Manchester, NH (US); Lius Alvarez, Lexington, MA (US); Mohammadali Safavieh, Nashua, NH (US)

(73) Assignee: Lung Biotechnology PBC, Silver Spring, MD (US)

(21) Appl. No.: 17/738,686

(22) Filed: May 6, 2022

Related U.S. Application Data
(60) Provisional application No. 63/185,299, filed on May 6, 2021.

(51) Int. Cl. B29C 64/129; B29C 64/264; B33Y 10/00; B33Y 70/00; B33Y 80/00; A61F 2/04; A61L 27/52; A61L 27/18

(52) U.S. Cl. CPC B29C 64/129; B29C 64/264; B33Y 10/00; B33Y 70/00; B33Y 80/00; A61F 2/04; A61L 27/52; A61L 27/18; B29K 2105/0002

(57) ABSTRACT

Embodiments of the present disclosure include methods of simultaneously manufacturing two or more hydrogel constructs (e.g., tubular hydrogel constructs). In some embodiments, the method comprises one or more of the following steps: providing a vat comprising a bio-ink composition containing one or more monomers and/or one or more polymers; applying electromagnetic radiation from an electromagnetic radiation source to cure a layer of the hydrogel constructs (e.g., tubular hydrogel constructs); and applying electromagnetic radiation from the electromagnetic radiation source one or more additional times to produce one or more additional layers of the hydrogel constructs (e.g., tubular hydrogel constructs).

### CROSS-REFERENCE TO RELATED APPLICATIONS

The present application claims priority to U.S. provisional application No. 63/185,299 filed May 6, 2021, which is incorporated herein by reference in its entirety.

### BACKGROUND

Compositions, including hydrogels, may be used to form objects used for biocompatible structures. These objects may be formed using three-dimensional (3D) printing techniques. Cells may be attached for practical applications such as synthetic organs.

### SUMMARY

Embodiments of this disclosure relate to a method of simultaneously manufacturing two or more tubular hydrogel constructs comprising: providing a vat comprising a bio-ink composition containing one or more monomers, one or more polymers, one or more UV absorbers, one or more photoinitiators, one or more natural or synthetic ECMs, and/or peptides; applying electromagnetic radiation from an electromagnetic radiation source to cure a layer of the tubular hydrogel constructs; and applying electromagnetic radiation from the electromagnetic radiation source one or more additional times to produce one or more additional layers of the tubular hydrogel constructs. In some embodiments, the electromagnetic radiation is UV radiation. In some embodiments, 10 or more tubular hydrogel constructs are simultaneously manufactured. In some embodiments, the vat further comprises a liquid that is immiscible with the bio-ink. In some embodiments, the bio-ink comprises a poly(ethylene glycol) di-(meth)acrylate polymer. In some embodiments, the bio-ink comprises at least one photoinitiator. In some embodiments, the bio-ink comprises DI water. In some embodiments, the bio-ink further comprises a UV dye, a protein, peptide, biologic, pharmaceutical compound, and/or extracellular matrices material. In some embodiments, the tubular hydrogel construct is substantially the same shape, size, and/or has the same relative dimensions of an organ or a fragment of an organ. In some embodiments, the organ or fragment of the organ comprises a vessel, trachea, bronchi, esophagus, ureter, renal tubule, bile duct, renal duct, renal tubules, bile duct, hepatic duct, nerve conduit, CSF shunt, larynx, or pharynx. In some embodiments, the vessel comprises a pulmonary artery, renal artery, coronary artery, peripheral artery, pulmonary vein, or renal vein. In some embodiments, the tubular hydrogel construct comprises a hemodialysis graft. In some embodiments, the tubular hydrogel construct permits endothelialization of an inner lumen of the tubular hydrogel construct and/or cellularization of the outer surface of the tubular hydrogel construct. In some embodiments, an inner lumen of the tubular hydrogel construct comprises a patterned surface. In some embodiments, the patterned surface includes patterning that permits unidirectional flow through the tube. In some embodiments, the tubular hydrogel construct comprises one or more bifurcation. In some embodiments, the hydrogel construct comprises a polymer selected from the group consisting of polymerized poly(ethylene glycol) di(meth)acrylate, polymerized poly(ethylene glycol) di(meth)acrylamide, polymerized poly(ethylene glycol) (meth)acrylate/(methacrylamide), poly(ethylene glycol)-block-poly(ε-caprolactone), polycaprolactone, polyvinyl alcohol, gelatin, methylcellulose, hydroxyethyl methyl cellulose, hydroxypropyl methyl cellulose, polyethylene oxide, polyacrylamides, polyacrylic acid, polymethacrylic acid, salts of polyacrylic acid, salts of polymethacrylic acid, poly(2-hydroxyethyl methacrylate), polylactic acid, polyglycolic acid, polyvinylalcohol, polyanhydrides such as poly(methacrylic) anhydride, poly(acrylic) anhydride, polysebasic anhydride, collagen, poly(hyaluronic acid), hyaluronic acid-containing polymers and copolymers, polypeptides, dextran, dextran sulfate, chitosan, chitin, agarose gels, fibrin gels, soy-derived hydrogels, alginate-based hydrogels, poly(sodium alginate), hydroxypropyl acrylate (HPA), lithium phenyl-2,4,6-trimethylbenzoylphosphinate (LAP), sodium phenyl-2,4,6-phenyl-2,4,6-trimethylbenzoylphosphinate (NaP), and combinations thereof.

Additional embodiments include a batch of tubular hydrogel constructs manufactured by the process of the above embodiments. In some embodiments, the tubular hydrogel constructs comprise different shapes

### FIGURES

FIG. 1A shows a cross sectional view of an embodiment of a plurality of hydrogel constructs. FIG. 1B shows a 45 degree angle view of the embodiment in FIG. 1A.

FIG. 2 shows a model for a printed tube.

FIG. 3 is a photograph of a printed tube.

FIG. 4 is a photograph of printed tubes.

FIGS. 5A-D are photographs of attachments of printed tubes to the modified tube attachment fixture.

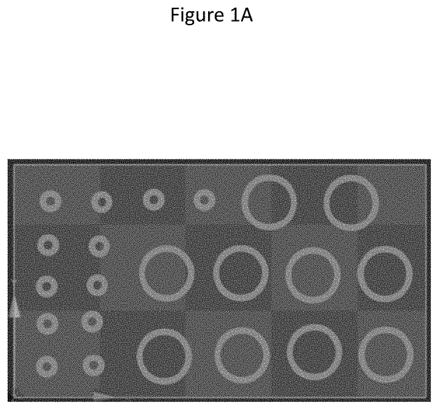

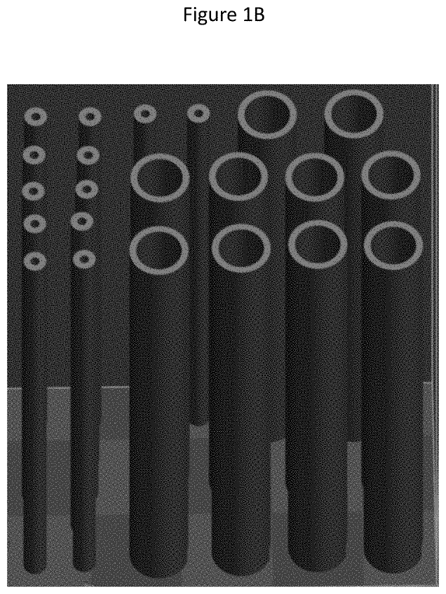

### DETAILED DESCRIPTION

As used herein, “3D printing” refers to any technique used to make a three-dimensional object using a digital model of that object. Exemplary 3D printing techniques include [insert].

As used herein, “printable ink” and “printable composition” refer to any composition that can be used to form an object using a 3D printing technique. A “bioink” is a printable ink that forms a material with one or more desired biocompatibility properties. For example, a bioink may contain one or more materials that facilitate adhesion and proliferation of desired cell types. The printed object may support primary cell and induced pluripotent stem cell attachment, proliferation, interactions, and spreading. In some cases, the bioink can be formed into a hydrogel. Compounds in the bioink may be selected or modified to incorporate chemical functionality, such as by chemical synthesis means. Chemical functionality may allow the incorporation of modified material as a component in the bioink. The modifications may allow chemical conjugation of a desired component. The desired component may maintain its cell interactive feature. Such incorporation may allow modulation of the printed object's mechanical properties without interfering with cell adhesion.

As used herein, “extracellular matrix” and “ECM” refer to natural and synthetic ECMs as well as one or more materials that constitute an ECM. For example, ECM can refer to a naturally-occurring ECM or an ECM made using synthetic techniques. ECM can also refer to one or more materials that constitute a naturally-occurring ECM, such as collagen (natural or synthetic). In some cases, “ECM material” will be used to refer to specific materials. ECM can be made using techniques, including 3D printing. The ECMs can be made using a hydrogel material.

As used herein, “extracellular matrix” and “ECM” refer to natural and synthetic ECMs as well as one or more materials that constitute an ECM. For example, ECM can refer to a naturally-occurring ECM or an ECM made using synthetic techniques. ECM can also refer to one or more materials that constitute a naturally-occurring ECM, such as collagen either natural or synthetic. In some cases, “ECM material” will be used to refer to specific materials. ECM can be made using techniques, including 3D printing. The ECMs can be made using a hydrogel material. ECM matrix material, such as collagen I, gelatin, elastin, and fibronectin, may be functionalized with methacrylate groups to enable incorporation into photo-crosslinkable hydrogels. Incorporation of ECM materials to other materials and objects, such as 3D printed materials, may increase biocompatibility and enable cell attachment and interaction within the materials and objects. The extent to which a material enables cell attachment can vary based on the amount of ECM material, the availability of binding sites on or within the material, the surface charge of the material, the polarity of the material, as well as the mechanical properties of the material.

As used herein, the terms “object,” “construct” and “article” may be used interchangeably and refer to items comprising the compositions of the invention.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention. When an embodiment is defined by one of these terms (e.g., “comprising”) it should be understood that this disclosure also includes alternative embodiments. Some of these embodiments may include “consisting essentially of” and “consisting of” for said embodiment.

As used herein, (meth)acrylate means methacrylate and/or acrylate.

As used herein, unless otherwise specified, “molecular weight” means a number-average molecular weight.

Unless specified, % refers to mass %.

The present application incorporates by reference in their entirety each of the following documents: (a) U.S. provisional application No. 63/185,293 filed May 6, 2021 titled “USE OF FUNCTIONALIZED AND NON-FUNCTIONALIZED ECMS, ECM FRAGMENTS, PEPTIDES AND BIOACTIVE COMPONENTS TO CREATE CELL ADHESIVE 3D PRINTED OBJECTS” and U.S. non-provisional and/or PCT application(s) under the same title filed on May 6, 2022; (b) U.S. provisional application No. 63/185,302 filed May 6, 2021 titled “MODIFIED 3D-PRINTED OBJECTS AND THEIR USES” and U.S. non-provisional and/or PCT application(s) under the same title filed on May 6, 2022; (c) U.S. provisional application No. 63/185,305 filed May 6, 2021 titled “PHOTOCURABLE REINFORCEMENT OF 3D PRINTED HYDROGEL OBJECTS” and U.S. non-provisional and/or PCT application(s) under the same title filed on May 6, 2022; (d) U.S. provisional application No. 63/185,300 filed May 6, 2021 titled “CONTROLLING THE SIZE OF 3D PRINTING HYDROGEL OBJECTS USING HDROPHILIC MONOMERS, HYDROPHOBIC MONOMERS, AND CROSSLINKERS” and U.S. non-provisional and/or PCT application(s) under the same title filed on May 6, 2022; (e) U.S. provisional application No. 63/185,298 filed May 6, 2021 titled “MICROPHYSIOLOGICAL 3-D PRINTING AND ITS APPLICATIONS” and U.S. non-provisional and/or PCT application(s) under the same title filed on May 6, 2022.

ECMs may be functionalized with methacrylate groups by substituting the lysine residue on the amine group with methacrylate anhydride(MAA). The degree of methacrylation of an ECM can be defined by the percentage of available amine groups which have been modified with MAA. A higher degree of methacrylation correlates with more MAA modified amine groups resulting in less free amine groups.

Embodiments of the present disclosure include methods of simultaneously manufacturing two or more hydrogel constructs (e.g., tubular hydrogel constructs). In some embodiments, the method comprises one or more of the following steps: providing a vat comprising a bio-ink composition containing one or more monomers and/or one or more polymers; applying electromagnetic radiation from an electromagnetic radiation source to cure a layer of the hydrogel constructs (e.g., tubular hydrogel constructs); and applying electromagnetic radiation from the electromagnetic radiation source one or more additional times to produce one or more additional layers of the hydrogel constructs (e.g., tubular hydrogel constructs).

An efficient technology among 3D printing technologies is a digital light process (DLP) method or stereolithography (SLA). In a 3D printer using the DLP or SLA method, the ink material is layered on a container or spread on a sheet, and a predetermined area or surface of the ink is exposed to ultraviolet-visible (UV/Vis) light that is controlled by a digital micro-mirror device or rotating mirror. In the DLP method, additional portions are repeatedly or continuously laid and each layer is cured until a desired 3D article is formed. The SLA method is different from the DLP method in that ink is solidified by a line of radiation beam. Other methods of 3D printing may be found in 3D Printing Techniques and Processes by Michael Degnan, December 2017, Cavendish Square Publishing, LLC, the disclosure of which is hereby incorporated by reference.

In some embodiments, the polymerization/curing of a layer of the hydrogel construct (e.g., tubular hydrogel construct) is performed at a vat temperature within the range of about 4° C. to about 37° C., e.g., at room temperature.

In some embodiments, the electromagnetic radiation is UV radiation. For example, the UV radiation may be suitable for UV-initiated polymerization, and the composition may include, e.g., a UV-initiator or photoinitiator compound that reacts and absorbs light at the range of 100-400 nm. Photoinitiators, may include, for example, benzophenone, phenyl bis (2,4,6-trimethylbenzoyl) phosphine oxide (BAPO), 2-hydroxy-2-methyl-1-phenyl-propan-1-one, 2-hydroxy-4′-(2-hydroxethoxy)-2-methylpropiophenone, 2,2′-azobis[2-methyl-n-(2-hydroxyethyl)propionamide], 2,2-dimethoxy-2-phenylacetophenone, diphenyl(2,4,6-trimethylbenzoyl)phosphine oxide, lithium phenyl(2,4,6- trimethylbenzoyl) phosphinate (LAP), and ethyl (2,4,6-trimethylbenzoyl) phenylphosphinate, and sodium phenyl-2,4,6-phenyl-2,4,6-trimethylbenzoylphosphinate (NaP).

In some embodiments, 10 or more hydrogel constructs (e.g., tubular hydrogel constructs) are simultaneously manufactured. For example, 10, 20, 30, 40, 50, 60, 70, 80, 90, 100, or more hydrogel constructs (e.g., tubular hydrogel constructs) may be simultaneously manufactured. The hydrogel constructs (e.g., tubular hydrogel constructs) that are simultaneously manufactured may be the same or different shape from one another.

The vat comprising a bio-ink composition may also contain other components, such as a liquid that is immiscible with the bio-ink. In some embodiments, the liquid that is immiscible with the bio ink is selected from one or more hydrophobic substance. For example, in some embodiments, the liquid that is immiscible is selected from mineral oil, butyl acetate, petroleum ether and mixtures thereof. In some embodiments, the mixture comprises about 25% (w/w) to about 50% (w/w) petroleum ether (e.g., about 25%, 30%, 35%, 40%, 45%, or 50% (w/w) petroleum ether). In some embodiments, the mixture comprises about 25% (w/w) to about 50% (w/w) butyl acetate (e.g., about 25, 30, 35, 40, 45, or 50% (w/w) petroleum ether). In some embodiments, the mixture comprises mineral oil, e.g., about 50% (w/w) to about 90% (w/w) mineral oil (e.g., about 50%, 55%, 60%, 65%, 70%, 75%, 80%, 85%, or 90% (w/w), or a range therein between). In some embodiments, the one or more hydrophobic substances comprises an oil with a viscosity at 25° C. of at least 5 cP (e.g., at least 5, 6, 7, 8, 9, 10, 11, 12, 13, 14, 15, 20, 25 cP, or a range therein between) and/or an organic solvent having a boiling point at STP above 100° C. (e.g., above 105, 110, 120, 130, 140, 150, 160, 200, 250, 300, 350, 400, 450, 500, 550, 600, 650° C., or a range therein between).

During printing, the hydrogel object may be immersed in liquid during the entire print duration. This immersion may prevent dehydration and provide buoyancy. During the methods embodied herein, the vat may from time to time be reloaded with additional components of the bio-ink and/or additional liquid that is immiscible with the bio-ink.

The bio-ink of the present embodiments is not particularly limited, and can be suitable to form, e.g., a composite structure made of one or more different polymerized monomers. Hydrogel materials that may be used in the invention may be known to those having ordinary skill in the art, as are methods of making the same. For example, a hydrogel as described in Caló et al., European Polymer Journal Volume 65, April 2015, Pages 252-267 may be used. In some embodiments, the hydrogel structure comprises a polymerized (meth)acrylate and/or (meth)acrylamide hydrogel. In some embodiments, the structure comprises a polymer comprising polymerized poly(ethylene glycol) di(meth)acrylate, polymerized poly(ethylene glycol) di(meth)acrylamide, polymerized poly(ethylene glycol) (meth)acrylate/(methacrylamide), poly(ethylene glycol)-block-poly(ε-caprolactone), polycaprolactone, polyvinyl alcohol, gelatin, methylcellulose, hydroxyethyl methyl cellulose, hydroxypropyl methyl cellulose, polyethylene oxide, polyacrylamides, polyacrylic acid, polymethacrylic acid, salts of polyacrylic acid, salts of polymethacrylic acid, poly(2-hydroxyethyl methacrylate), polylactic acid, polyglycolic acid, polyvinylalcohol, polyanhydrides such as poly(methacrylic) anhydride, poly(acrylic) anhydride, polysebasic anhydride, collagen, poly(hyaluronic acid), hyaluronic acid-containing polymers and copolymers, polypeptides, dextran, dextran sulfate, chitosan, chitin, agarose gels, fibrin gels, soy-derived hydrogels, alginate-based hydrogels, poly(sodium alginate), hydroxypropyl acrylate (HPA), lithium phenyl-2,4,6-trimethylbenzoylphosphinate (LAP) and combinations thereof. In some embodiments, the M_{w} of the hydrogel polymer is about 400 Da, 500 Da, 600 Da, 700 Da, 800 Da, 900 Da, 1000 Da, 1100 Da, 1200 Da, 1300 Da, 1400 Da, 1500 Da, 1600 Da, 1700 Da, 1800 Da, 1900 Da, 2000 Da, 2100 Da, 2200 Da, 2300 Da, 2400 Da, 2500 Da, 2600 Da, 2700 Da, 2800 Da, 2900 Da, 3000 Da, 3100 Da, 3200 Da, 3300 Da, 3400 Da, 3500 Da, 3600 Da, 3700 Da, 3800 Da, 3900 Da, 4000 Da, 4100 Da, 4200 Da, 4300 Da, 4400 Da, 4500 Da, 4600 Da, 4700 Da, 4800 Da, 4900 Da, 5000 Da, 5100 Da, 5200 Da, 5300 Da, 5400 Da, 5500 Da, 5600 Da, 5700 Da, 5800 Da, 5900 Da, 6000 Da, 6100 Da, 6200 Da, 6300 Da, 6400 Da, 6500 Da, 7000 Da, 7500 Da, 8000 Da, 8500 Da, 9000 Da, 9500 Da, 10000 Da, 15000 Da, or 20000 Da. In some embodiments, the bio-ink may include two or more hydrogel polymers each having a distinct molecular weight.

In some embodiments, a concentration of hydrogel polymer(s) in the bio-ink may be from about 5% to about 50% or from about 10% to about 40% or from about 15% to about 30%, such as about 20%, or any value or subrange within these ranges.

The dimensions of the hydrogel constructs (e.g., tubular hydrogel constructs) are not particularly limited, and may be altered depending on the application. In some embodiments, the hydrogel constructs (e.g., tubular hydrogel constructs) includes a plurality of layers, which have a thickness of 200 μm to 500 μm. In some embodiments, the tubular hydrogel constructs have a wall thickness of up to about 1 mm, about 2 mm, about 3 mm, about 4 mm or about 5 mm. For example, the wall thickness may be about 0.05 mm, about 0.1 mm, about 0.2 mm, about 0.3 mm, about 0.4 mm, about 0.5 mm, about 0.6 mm, about 0.7 mm, about 0.8 mm, about 0.9 mm, about 1 mm, about 1.1 mm, about 1.2 mm, about 1.3 mm, about 1.4 mm, about or about 1.5 mm (or a range therein between). In some embodiments, the tubular hydrogel constructs have a length or up to about 250 mm (e.g., about 10 mm, about 20 mm, about 30 mm, about 40 mm, about 50 mm, about 60 mm, about 70 mm, about 80 mm, about 90 mm, about 100 mm, about 110 mm, about 120 mm, about 130 mm, about 140 mm, about 150 mm, about 160 mm, about 170 mm, about 180 mm, about 190 mm, about 200 mm, about 210 mm, about 220 mm, about 230 mm, about 240 mm, about or 250 mm (or a range therein between)).

In some embodiments, the bio-ink comprises a poly(ethylene glycol) di-(meth)acrylate polymer. In some embodiments, the poly(ethylene glycol) di-(meth)acrylate polymer has a weight average molecular weight (M_{w}) of about 400 to about 20,000 (e.g., about 400, 500, 100, 1500, 2000, 2500, 3000, 3500, 4000, 4500, 5000, 5500, 6000, 6500, 7000, 7500, 8000, 8500, 9000, 9500, 10000, 10500, 11000, 11500, 12000, 12500, 13000, 13500, 14000, 14500, 15000, 15500, 16000, 16500, 17000, 17500, 18000, 18500, 19000, 19500 or 20000, or a range therein between). In some embodiments, the bio-ink may include two or more poly(ethylene glycol) di-(meth)acrylate polymers each having a distinct molecular weight.

In some embodiments, a concentration of poly(ethylene glycol) di-(meth)acrylate polymer(s) in the bio-ink may be from about 5% to about 50% or from about 10% to about 40% or from about 15% to about 30%, such as about 20%, or any value or subrange within these ranges.

In some embodiments, the bio-ink comprises one or more of hydroxy C₁₋₂ alkyl (meth)acrylates, poly(alkylene oxide) alkyl ether (meth)acrylates, N-hydroxy C₁₋₂ alkyl (meth)acrylamides, a\ poly(ethylene glycol) methyl ether acrylate (PEGMEA), poly(ethylene glycol) methyl ether methacrylate, poly(propylene glycol) methyl ether acrylate, poly(propylene glycol) methyl ether methacrylate, hydroxyethyl acrylate (HEA), N-hydroxyethyl acrylamide (HEAA), hydroxyethyl methacrylate, hydroxypropyl acrylate (HPA 3-Hydroxypropyl acrylate and/or 2-Hydroxypropyl acrylate), hydroxypropylmethacrylate, hydroxybutyl acrylate (HBA), hydroxybutyl methacrylate, poly(alkylene oxide) di(meth)acrylates, diethylene glycol di(meth)acrylate, tetraethylene glycol di(meth)acrylate, N,N′-methylenebis(acylamide), (poly)lactic acid di(meth)acrylate, (poly)glycolic acid di(meth)acrylate, (poly)lactic-coglycolide di(meth)acrylate, (poly)caprolactone di(meth)acrylate, (poly)dioxanone di(meth)acrylate, (poly)fumarate di(meth)acrylate, (caboxy)(methyl)cellulose di(meth)acrylate, hyaluronic acid di(meth)acrylate, heparan sulfphate di(meth)acrylate, dextran di(meth)acrylate, alginate di(meth)acrylate, pectin di(meth)acrylate, or collagen di(meth)acrylate or mixtures thereof.

In some embodiments, the bio-ink may comprises one or more poly(ethylene glycol) di-(meth)acrylate polymer(s) and one or more additional polymers, such as alginate-based hydrogel. In some embodiments, a concentration of the one or more additional polymers in the bioink may be from about 0.5% to about 10% or from about 1% to about 8% or from about 1.5% to about 5%, such as about 2.5%, or any value or subrange within those ranges.

In some embodiments, the bio-ink comprises further comprises a photo initiator. The photo initiator is not particularly imitated, and examples of suitable photo initiators include lithium phenyl-2,4,6-trimethylbenzoylphosphinate (LAP), Trimethylbenzoyl based photoinitiators, diphenyl(2,4,6-trimethylbenzoyl)phosphine oxide (TPO nanoparticle) Irgacure class of photoinitiators, ruthenium, riboflavin, sodium phenyl-2,4,6-phenyl-2,4,6-trimethylbenzoylphosphinate (NaP), or mixtures thereof. In some embodiments, a concentration of a photo-initiator in the bio-ink may be from about 0.1% to about 5% or from about 0.2% to about 3% or from about 0.5% to about 2%, such as about 1%, or any value or subrange within those ranges.

In some embodiments, the bio-ink further comprises a solvent, such as water. In some embodiments the water is deionized. In certain embodiments, the bio-ink comprises about 50 to about 90% DI water (e.g., about 50%, 55%, 60%, 65%, 70%, 75%, 80%, 85%, 90% DI water, or a range therein between).

In some embodiments, the bio-ink further comprises a UV dye, a protein, peptide, biologic, pharmaceutical compound, and/or extracellular matrices material. In some embodiments the peptides are selected from RGD, KQAGDV, YIGSR, REDV, IKVAV, RNIAEIIKDI, KHIFSDDSSE, VPGIG, FHRRIKA, KRSR, APGL, VRN, AAAAAAAAA, GGLGPAGGK, GVPGI, LPETG(G)n, and IEGR. Other examples of suitable additional components include ECM or ECM-like material such as amino acid sequence sensitive to a protease. The protease may be selected from Arg-C proteinase, Asp-N endopeptidase, BNPS-Skatole, Caspase 1-10, Chymotrypsin-high specificity (C-term to [FYW], not before P), Chymotrypsin-low specificity (C-term to [FYWML], not before P), Clostripain (Clostridiopeptidase B), CNBr, Enterokinase, Factor Xa, Formic acid, Glutamyl endopeptidase, GranzymeB, Hydroxylamine, Iodosobenzoic acid, LysC, Neutrophil elastase, NTCB (2-nitro-5-thiocyanobenzoic acid), Pepsin, Proline-endopeptidase, Proteinase K, Staphylococcal peptidase I, Thermolysin, Thrombin and Trypsin. In some embodiments, a concentration of a UV dye in the bio-ink may be from about 0.02% to about 2% or from about 0.03% to about 1.5% or from about 0.05% to about 1%, such as about 0.2%, or any value or subrange within those ranges.

In some embodiments, the hydrogel scaffold remains immersed or submerged (or partially immersed) in the liquid that is immiscible with the bio ink during the method. In some embodiments, the hydrogel scaffold is submerged in the container. In some embodiments, the hydrogel scaffold is submerged in the container. In some embodiments, the method further comprising adding the liquid that is immiscible with the bio-ink to replace at least a portion of the bio-ink consumed or otherwise lost during the printing. In some embodiments, the liquid that is immiscible with the bio-ink is positioned in the container to prevent the evaporation of the bio-ink.

In some embodiments the hydrogel construct (e.g., tubular hydrogel construct) is substantially the same shape, size, and/or has the same relative dimensions of an organ or a fragment of an organ. For example, the organ or fragment of the organ may comprise a vessel, trachea, bronchi, esophagus, ureter, renal tubule, bile duct, renal duct, renal tubules, bile duct, hepatic duct, nerve conduit, CSF shunt, larynx, or pharynx. For example, in some embodiments, the hydrogel constructs (e.g., tubular hydrogel constructs) described herein are formed into a structure that mimics or replicates a portion of the architecture of the lung, such as by using 3D printing techniques. The hydrogel constructs (e.g., tubular hydrogel constructs) can be used to form a scaffold for adhesion and growth of cells resulting in a structure that has one or more desired properties of an organ, such as a structure that can be perform the gas exchange functions of a lung. These objects can comprise a hydrogel. The organ or portion of an organ can be a human lung in a preferred embodiment.

In some embodiments, the tubular hydrogel construct comprises a hemodialysis graft. In some embodiments, the tubular hydrogel construct permits endothelialization of an inner lumen of the tubular hydrogel construct and/or cellularization of the outer surface of the tubular hydrogel construct. In some embodiments, an inner lumen of the tubular hydrogel construct comprises a patterned surface. In some embodiments, the patterned surface includes patterning that permits unidirectional flow through the tube. In some embodiments, the tubular hydrogel construct comprises one or more bifurcation.

In some embodiments, in order to produce a tubular construct, the bio-ink may be exposed to electromagnetic radiation, such as UV radiation. In some embodiments, intensity of electromagnetic radiation, such as UV radiation, may be from 1 mW/cm² to 100 mW/cm² or from 2 mW/cm² to 80 mW/cm² or from 5 mW/cm² to 50 mW/cm² or any value or subrange within those ranges. In some embodiments, a time of exposure to electromagnetic radiation, such as UV radiation, may be from 0.1 sec to 100 sec or from 0.1 sec to 50 sec or from 0.2 sec to 30 sec or any value or subrange within those ranges.

In some embodiments, the tubular hydrogel construct may be attached to a pump, such as a peristaltic pump. For such attachment, an adhesive such as a glue, which may be, for example, a cyanoacrylate glue, may be used.

The examples described herein are illustrative of the present invention and are not intended to be limitations thereon. Different embodiments of the present invention have been described according to the present invention. Many modifications and variations may be made to the techniques described and illustrated herein without departing from the spirit and scope of the invention. Accordingly, it should be understood that the examples are illustrative only and are not limiting upon the scope of the invention.

### EXAMPLE 1

### 3D Printing with PolyEthylene Glycol-DiAcrylate (PEG-DA)

A PEG-DA 6k solution was prepared using PEG-DA 6k; LAP; UV386a (UV dye from QCR Solutions Corp.) and DI Water.

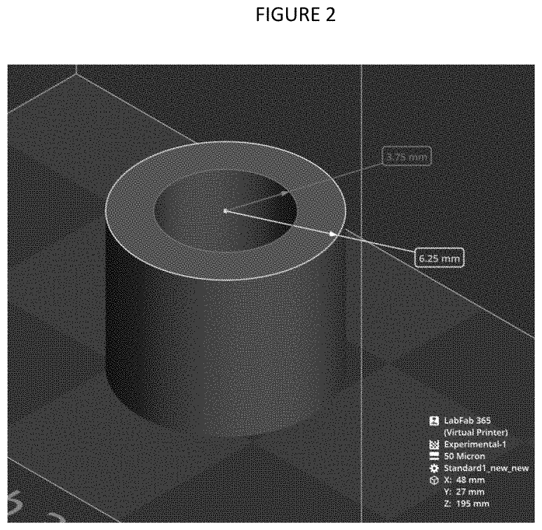

A tube was 3D-printed with the PED-GA 6k solution. A model for the printed tube had a 12.5 mm outer diameter (OD), 7.5 mm inner diameter (ID) and 10 mm height, see FIG. 2.

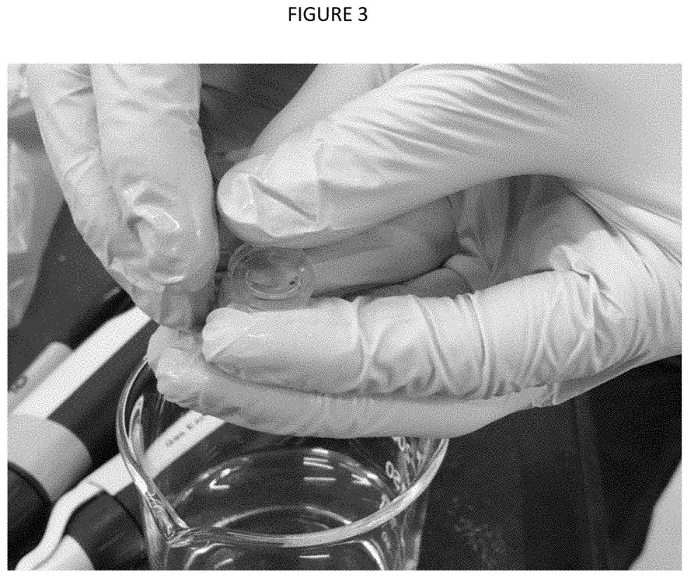

FIG. 3 is a photograph of the printed tube.

### EXAMPLE 2

To print a set of long 5 mm ID tubes having 1.5 mm and 2 mm wall thicknesses. Based on this, large numbers of the 5 mm ID tubes of the 2 wall thicknesses of interest were printed.

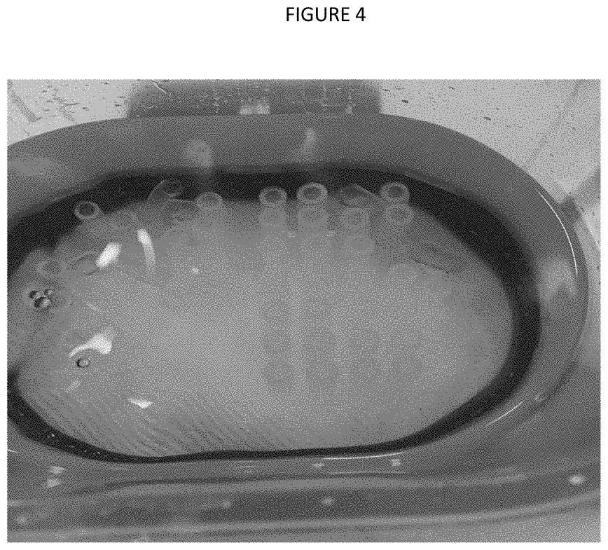

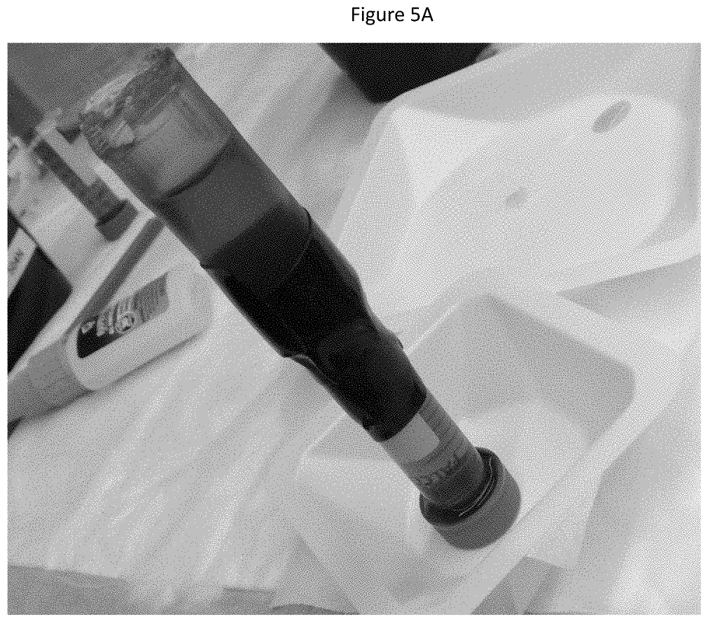

FIG. 4 is a photograph of the printed tubes.

### EXAMPLE 3

A short (3-10 cm) 3-D printed tube was fixed at both ends to tubing and attached to a peristaltic pump. Fixation was be achieved using medical grade mesh and cyanoacrylate glue. Following curing, fluid was passed through the tube for as long as possible until leaking was observed.

Seven of eight tubes printed successfully. Unsuccessful tube printed like a spiral in several pieces.

After the print was completed the tubes were rinsed in tap water for 5 minutes and soaked in phosphate-buffered saline (PBS) for 45 minutes.

Extra ink material was poured back into an amber jug.

Then two of the tubes were tested for attachment.

The first tube was successfully connected to tubing with OD of 14 mm using DERMABOND® PRINEO® Skin Closure System. Instructions were followed as written. On both sides. 60 sec cure time was used. Then sample was torqued by accident and ruptured in the middle. Ends stayed attached. Sample was filled with tap water that was dyed red with food coloring.

The second tube was attached using DERMABOND® PRINEO® Skin Closure System following modified instructions. This time, two wraps of polyethylene (PE) mesh were placed on each side. Wraps were staggered rather than overlapping completely, giving generous coverage to the seam between the sample end and the tubing.

This configuration was run on the CP peristaltic pump at max flow rate over the weekend at max flow rate. Sample was submerged in DI water so it would not dry out over the weekend, and so that leakage could be assessed by noting a color change in the water of the bath.

The connections were still intact and the flow rate was turned down from 170 to 70 ml/min for about three hours. It was turned down because the pump motor sounded off. Then it was turned back up to 120 ml/min to give connections a higher challenge.

Sample continued through the whole work day without leaking. Water looks tinged pink as if dyed water diffused through sample. Sample continues to run overnight.

Sample connections remains intact overnight to next morning. Water bath still looks tinged pink, but no leaks are apparent.

### EXAMPLE 4

To try connecting previously printed tubes to a modified connector. Previous model of a connector did not have room to thread coupling around the swollen printed tube. The design has been modified for increased wall thickness due to swelling. Additionally, once the tube connector piece is in place it was further sealed.

### Materials

Previously printed 2.5 cm tube sections. Samples stored in PBS in dark drawer.

Loctite Pro Line Marine Fast Cure Adhesive Sealant. E-ZFuse Tape—Self-fusing, waterproof, airtight seal—Black Silicone Tape. FiberFix—Flex Patch. Rapid Fuse DAP. Cyanoacrylate glue—Rapid Fuse. Rubber Cement.

### Methods & Results

In each case, excess buffer was gently dabbed off of the tube surface. The product was applied as indicated on the instructions provided. Press fit onto the newly designed tube fitting. Sample would not stay in place. Kept slipping off. After holding it in place for short duration, tube split.

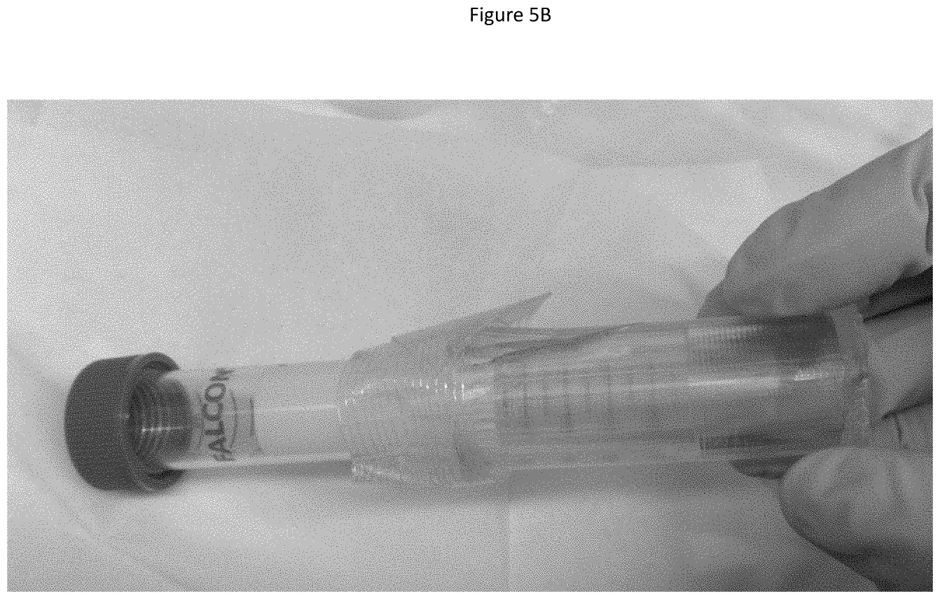

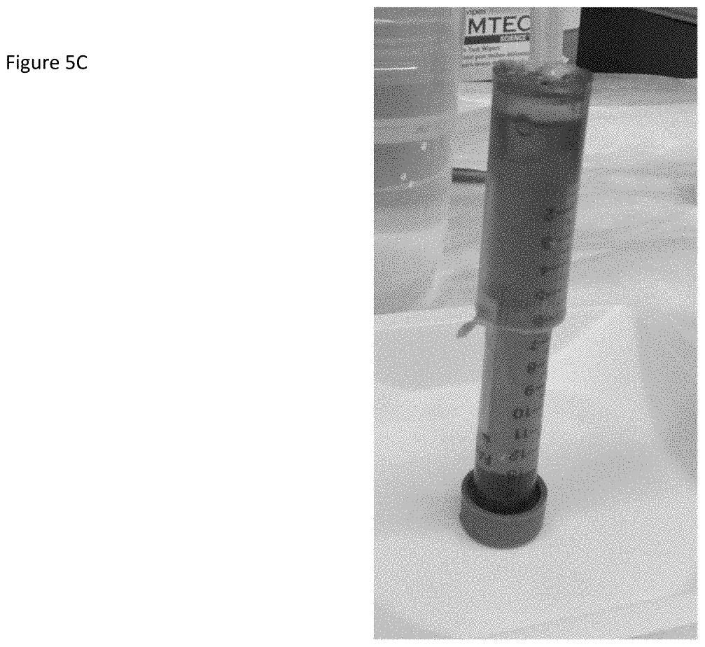

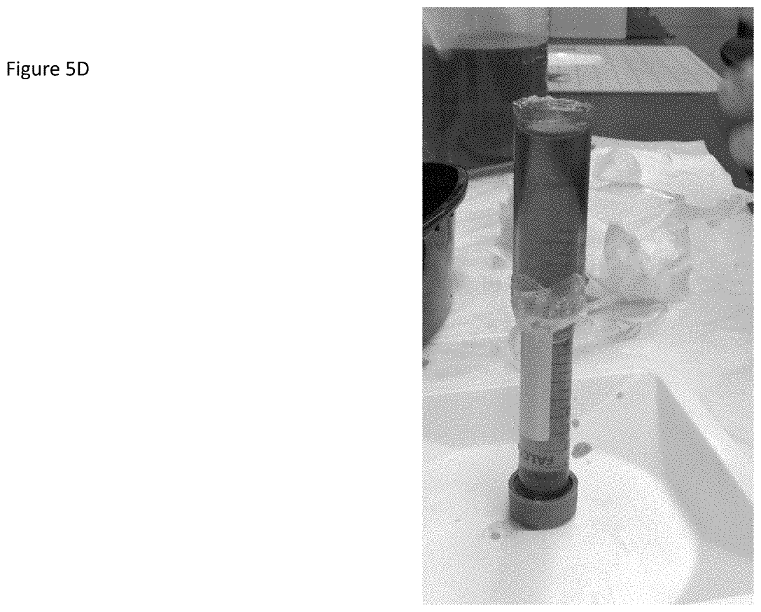

### Application of Silicone Tape—EZ Fuse—FIG. 5A

Initial application to the printed structure was not successful. Tape sticks quite well to itself and actually seemed to create a seal in later attempts. It is hard to get to fully conform to the shape of the container without applying significant stretching and pressure which will crack our printed structure. It is also impossible to see through the tape to see what is happening underneath. However, it was one of the better candidates and should not be discounted.

Fiber Flex Patch—FIG. 5B

Very hard to work with. Sticks to gloves and itself. Did not conform to the sample as well as the black tape did. Sample slipped right out from under the taped section.

### Clear Silicone Sealant

Applied to the surface pretty well and filled the gaps in the crack of the tube. Curing time ˜24 hours. Did not set up in time to be useful. Pulled cleanly off of the material surface.

### Rapid Fuse Foam

Seemed to stick pieces together, however very messy to use, hard to control application area. Did not firm up in time to be useful.

### Cyanoacrylate Glue. FIGS. 5C-D

Affixed two scrap pieces together. Definitely showed adherence after ˜30 sec. Glued tube to a 15 mL Falcon tube. Initially thought that it successfully sealed the connection, but later realize that it may have been only the press fit that was sealing it, see FIG. 5C.

Collar of glue with no attachment to the printed piece. Still no leaking observed.

In order to address this, surgical mesh was applied and the glue overlayed on the mesh (basically as surgical instructions describe, substituting the over the counter glue for Dermabond), see FIG. 5D. Attached this construct to the end of a piece of tubing mounted in a peristaltic pump. Was able to flow water through at a significant flow rate.

Attempted to attach both ends to the pump in order to continuously circulate flow through the part. Initial attachment seemed to go well, but as the printed part flexed slightly it cracked completely into 2 pieces. Very brittle, could not withstand any displacement. May be due to the age of the printed pieces or inherent brittleness of the material.

Attempted to reattach a new piece. There may have been a small gap in the mesh material—minimal mesh was used to try to preserve the small amounts that were remaining. It was possible to start the pumping process, but leaking started quickly at the interface of the tube and the printed piece.

After circulating a small volume of water, tried to remove the printed tube from the plastic tubing. Glue and mesh held so strongly that the printed piece broke rather than sliding off of the tubing.

### Conclusions

1. Surgical mesh combined with cyanoacrylate glue looks to be a viable option for attaching the printed material to a pump.

2. Silicone tape may be a backup approach.

As used herein, the singular terms “a,” “an,” and “the” may include plural referents unless the context clearly dictates otherwise. Thus, for example, reference to an object may include multiple objects unless the context clearly dictates otherwise.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. When referring to a first numerical value as “substantially” or “about” the same as a second numerical value, the terms can refer to the first numerical value being within a range of variation of less than or equal to ±10% of the second numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

While the disclosure has been described with reference to the specific embodiments thereof, it should be understood by those skilled in the art that various changes may be made and equivalents may be substituted without departing from the true spirit and scope of the disclosure as defined by the appended claim(s). In addition, many modifications may be made to adapt a particular situation, material, composition of matter, method, operation or operations, to the objective, spirit and scope of the disclosure. All such modifications are intended to be within the scope of the claim(s) appended hereto. In particular, while certain methods may have been described with reference to particular operations performed in a particular order, it will be understood that these operations may be combined, sub-divided, or re-ordered to form an equivalent method without departing from the teachings of the disclosure. Accordingly, unless specifically indicated herein, the order and grouping of the operations is not a limitation of the disclosure.

All of the publications, patent applications and patents cited in this specification are incorporated herein by reference in their entirety

1. A method of simultaneously manufacturing two or more tubular hydrogel constructs comprising:
providing a vat comprising a bio-ink composition containing one or more monomers and/or one or more polymers;
applying electromagnetic radiation from an electromagnetic radiation source to cure a layer of the tubular hydrogel constructs; and
applying electromagnetic radiation from the electromagnetic radiation source one or more additional times to produce one or more additional layers of the tubular hydrogel constructs.

2. The method of claim 1, wherein the bio-ink composition comprises monomers.

3. The method of claim 1, wherein the bio-ink composition comprises one or more polymers.

4. The method of claim 1, wherein the electromagnetic radiation is UV radiation.

5. The method of claim 1, wherein 10 or more tubular hydrogel constructs are simultaneously manufactured.

6. The method of claim 1, wherein the vat further comprises a liquid that is immiscible with the bio-ink.

7. The method of claim 1, wherein the bio-ink composition comprises a poly(ethylene glycol) di-(meth)acrylate polymer.

8. The method of claim 1, wherein the bio-ink composition comprises at least one photoinitiator.

9. The method of claim 1, wherein the bio-ink composition comprises DI water.

10. The method of claim 1, wherein the bio-ink composition further comprises a UV dye, a protein, peptide, biologic, pharmaceutical compound, and/or extracellular matrices material.

11. The method of claim 1, wherein the tubular hydrogel construct is substantially the same shape, size, and/or has the same relative dimensions of an organ or a fragment of an organ.

12. The method of claim 11, wherein the organ or fragment of the organ comprises a vessel, trachea, bronchi, esophagus, ureter, renal tubule, bile duct, renal duct, renal tubules, bile duct, hepatic duct, nerve conduit, CSF shunt, larynx, or pharynx.

13. The method of claim 12, wherein the vessel comprises a pulmonary artery, renal artery, coronary artery, peripheral artery, pulmonary vein, or renal vein.

14. The method of claim 1, wherein the tubular hydrogel construct comprises a hemodialysis graft.

15. The method of claim 1, wherein the tubular hydrogel construct permits endothelialization of an inner lumen of the tubular hydrogel construct and/or cellularization of the outer surface of the tubular hydrogel construct.

16. The method of claim 1, wherein an inner lumen of the tubular hydrogel construct comprises a patterned surface.

17. The method of claim 16, wherein the patterned surface includes patterning that permits unidirectional flow through the tube.

18. The method of claim 1, wherein the tubular hydrogel construct comprises one or more bifurcation.

19. The method of claim 1, wherein the hydrogel construct comprises a polymer selected from the group consisting of polymerized poly(ethylene glycol) di(meth)acrylate, polymerized poly(ethylene glycol) di(meth)acrylamide, polymerized poly(ethylene glycol) (meth)acrylate/(methacrylamide), poly(ethylene glycol)-block-poly(ε-caprolactone), polycaprolactone, polyvinyl alcohol, gelatin, methylcellulose, hydroxyethyl methyl cellulose, hydroxypropyl methyl cellulose, polyethylene oxide, polyacrylamides, polyacrylic acid, polymethacrylic acid, salts of polyacrylic acid, salts of polymethacrylic acid, poly(2-hydroxyethyl methacrylate), polylactic acid, polyglycolic acid, polyvinylalcohol, polyanhydrides such as poly(methacrylic) anhydride, poly(acrylic) anhydride, polysebasic anhydride, collagen, poly(hyaluronic acid), hyaluronic acid-containing polymers and copolymers, polypeptides, dextran, dextran sulfate, chitosan, chitin, agarose gels, fibrin gels, soy-derived hydrogels, alginate-based hydrogels, poly(sodium alginate), hydroxypropyl acrylate (HPA), lithium phenyl-2,4,6-trimethylbenzoylphosphinate (LAP) and combinations thereof.

20. A batch of tubular hydrogel constructs manufactured by the process of claim 1.

21. The batch of claim 20, wherein the tubular hydrogel constructs comprise different shapes.
